(19) United States
(12) Patent Application Publication
(10) Pub. No.: US 2021/0298836 A1
(43) Pub. Date: Sep. 30, 2021

(54) HOLOGRAPHIC TREATMENT ZONE MODELING AND FEEDBACK LOOP FOR SURGICAL PROCEDURES

(71) Applicant: MEDIVIEW XR, INC., Cleveland, OH (US)

(72) Inventors: John Black, Bowling Green, OH (US); Mina S. Fahim, New Brighton, MN (US)

(21) Appl. No.: 17/213,636

(22) Filed: Mar. 26, 2021

Related U.S. Application Data
(60) Provisional application No. 63/000,408, filed on Mar. 26, 2020.

(51) Int. Cl. A61B 34/20; A61B 90/00; G03H 1/00

(52) U.S. Cl. CPC A61B 34/20; A61B 90/36; A61B 2090/366; A61B 2034/2065; A61B 2090/365; G03H 1/0005

(57) ABSTRACT

Performance of a medical procedure on an anatomical site can include acquiring a holographic image dataset from a patient. An instrument can be tracked using a sensor to provide a tracked instrument dataset and the holographic image dataset and the tracked instrument dataset can be registered with the patient. A hologram can be rendered based on the holographic image dataset from the patient for viewing by the user and to generate a feedback based on the holographic image dataset from the patient and the tracked instrument dataset. Performance of a portion of the medical procedure on the patient can occur while the user views the patient and the hologram with an augmented reality system, where the user can employ the augmented reality system for visualization, guidance, and/or navigation of the instrument during the medical procedure in response to the feedback.

### CROSS-REFERENCE TO RELATED APPLICATIONS

This application claims the benefit of U.S. Provisional Application No. 63/000,408, filed on Mar. 26, 2020. The entire disclosure of the above application is incorporated herein by reference.

### FIELD

The present technology relates to holographic augmented reality applications and, more particularly, medical applications employing holographic augmented reality.

### INTRODUCTION

This section provides background information related to the present disclosure which is not necessarily prior art.

Image-guided surgery has become standard practice for many different procedures, such as structural heart repairs. In particular, holographic visualization is an emerging trend in various surgical settings. Holographic visualizations leverage spatial computing, holography, and instrument tracking to produce a coordinate system accurately registered to a patient's anatomy. Tracking the instrument and having a coordinate system registered to the patient allows for a user (e.g., a surgeon or other medical practitioner) to utilize holographic visualizations to perform image-guided surgery. Undesirably, such systems do not presently track the relationship between the tracked instrument and the coordinate system registered to the patient's anatomy. For instance, the user does not receive predictive contextual data insights based on interaction of the tracked instrument with the patient anatomy.

There is a continuing need for a visualization and guidance system and method for performing a medical procedure, including the provision of real-time contextual data in the form of feedback. Desirably, the system and method would provide predictive real-time simulations based on the interaction between the tracked instrument and the patient.

### SUMMARY

In concordance with the present technology, ways of providing visualization and guidance in performing a surgical procedure include use of real-time contextual data in the form of one or more types of feedback, which can further include predictive real-time simulations based on an interaction between a tracked instrument and the anatomy of a patient, have been surprisingly discovered.

Systems and methods are provided for holographic augmented reality visualization and guidance in performing a medical procedure on an anatomical site of a patient by a user. Included are an augmented reality system, a tracked instrument having a sensor, an image acquisition system configured to acquire a holographic image dataset from the patient, and a computer system having a processor and a memory. The computer system can be in communication with the augmented reality system, the tracked instrument, and the image acquisition system. The image acquisition system can be used to acquire the holographic image dataset from the patient. The computer system can be used to track the tracked instrument using the sensor to provide a tracked instrument dataset. The computer system can be used to register the holographic image dataset and the tracked instrument dataset with the patient. The augmented reality system can be used to render a hologram based on the holographic image dataset from the patient for viewing by the user. The augmented reality system can be used to generate a feedback based on the holographic image dataset from the patient and the tracked instrument dataset. The user can perform a portion of the medical procedure on the patient while viewing the patient and the hologram with the augmented reality system. The user accordingly employs the augmented reality system for at least one of visualization, guidance, and navigation of the tracked instrument during the medical procedure in response to the feedback.

Aspects of the present technology enable certain functionalities having particular benefits and advantages in performance of the medical procedure. In particular, when a tracked instrument trajectory is displayed via a holographic or virtual needle guide, feedback can be provided to the user performing the medical procedure. For example, if the projected trajectory of the tracked instrument is in an optimal position, the holographic coordinate system can generate feedback in the form of audio or visual feedback indicating the optimal position is recognized and/or that the procedure can proceed to a next step. Oppositely, if the tracked instrument is going to interact with or affect a non-target structure, the feedback can alert the user of a potentially undesirable or unplanned outcome or step.

The present technology can also provide modeling for predictive outcome feedback depending on surgery-specific details from a particular interventional procedure. For example, ablation and drug therapies can employ specific parameters and/or doses depending on a type of tumor being treated, as well as the surrounding anatomy, including blood vessels. The present technology can use real-time measurement of distances of not only the tracked instrument, but also the adjustable volume, power, or type of therapy to be delivered to the subject tumor, heart, or lesions, which are known to influence broader clinical outcomes. The present systems and methods of using such systems as provided herein can therefore notify the user (e.g., surgeon) that a blood vessel, bile duct, or other structure is in a planned ablation zone, which could potentially lead to negative side effects with respect the planned medical procedure. Instead, the present technology can allow the user to either change the intensity of the therapy to be delivered, or alternatively, change the patient post procedural care and discharge planning based on an expected negative side effect secondary to the treatment. For example, where the system generates feedback to the user that a bile duct is within an ablation zone, and should not be subject to the ablation procedure, this data insight can be reflected in an operative report stating that a portion of the tumor was not ablated. Subsequently, using such contextual data insights, the user can recommend subsequent medical treatment, such as high precision proton therapy or other non-invasive methods, to complete a desired medical treatment based on an objective analysis of the procedure.

The present systems and methods can be used in various ways to provide visualization and guidance in performing a medical procedure. Non-limiting examples of various applicable medical procedures that can use the present technology include the following: (1) holographic modeling of microwave, radiofrequency, cryo and irreversible electroporation (IRE), high intensity focused ultrasound in bone and soft tissue; (2) holographic modeling of a skin lesion or tumor for the delivery of oncolytic or chemotherapy drugs to kill a tumor, predictive diffusion zone based on a tissue type, agent being delivered, and volume of agent delivered; (3) intracardiac mapping for electrophysiology ablation therapies such cryo and radiofrequency; (4) holographic mapping and pacing of a heart for mapping of an ablation zone of pulmonary veins and cardiac substrate, where contextual data insights can alert the user of an expected outcome at future time points based on extent of the ablation procedure to weigh risk versus reward in other indicated procedures; (5) orthopedic pediatric deformity correction procedures to allow for novel methods of planning osteogenesis distraction limb lengthening and center of rotation of angulation (CORA) centric and perpendicular procedures, including holographic identification of the mean axis of deviation and angulation to assist in planning and predicting the new alignment of the limbs to ensure the center of weight is aligned to a proper or desired anatomical position; (6) derotational osteotomy procedures to provide contextual data of overall incremental adjustments for proper staging of care treatment plans and to prevent injury of soft tissue with acute corrections, where holographic visualization, instrument tracking, and warnings are provided for piriformis fossa entry in pediatric femur fractures for avoidance of disrupting lateral circumflex artery, including use of holographic visualization for embolization procedures to ensure all blood supply to a tumor or lesion has been eliminated and end user feedback is provided if key supply or auxiliary vessels are still viable based on the dosage and location of embolization therapy; (7) visualization and predictive relief in spinal cord stimulation and peripheral nerve ablation therapies for pain management therapies, including visualization and localization of nerve and intervention points to assess treatment time based on scar profile, as well as collagen content size of the nerve being treated; (8) neurosurgical and spine procedures, including angulation calculation determined holographically for closed feedback loop for placing a pedical screw, feedback loop for stress profiles of spine support constructs for stress riser identification, and predictive yield points based on spine implant repetitive cycle testing and yield point data; and (9) structural heart prosthesis alignment and predictability for prevention of backflow from optimal implant placement and relationships of other anatomies.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a schematic illustration of a system for holographic augmented reality visualization and guidance in performing a medical procedure on an anatomical site of a patient by a user, depicting an augmented reality system, a tracked instrument, a computer system, a first image acquisition system, and a second image acquisition system in communication with one another via a computer network, in accordance with an embodiment of the present technology.

FIG. 2 is a schematic illustration of the tracked instrument as provided in the system of FIG. 1, in accordance with an embodiment of present technology.

FIG. 3 is a flowchart showing a process for performing a medical procedure using a holographic augmented reality visualization and guidance system, according to an embodiment of the present technology, in accordance with an embodiment of the present technology.

FIG. 4 is a schematic illustration of system components and process interactions showing ways to provide holographic augmented reality visualization and guidance in performing a medical procedure on an anatomical site of a patient by a user, in accordance with an embodiment of the present technology.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as can be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise.

Unless otherwise defined, all technical terms used herein have the same meaning as commonly understood by one of ordinary skill in the art to which the present disclosure pertains.

As used herein, the terms “a” and “an” indicate “at least one” of the item is present; a plurality of such items can be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that can arise from ordinary methods of measuring or using such parameters.

Although the open-ended term “comprising,” as a synonym of non-restrictive terms such as including, containing, or having, is used herein to describe and claim embodiments of the present technology, embodiments can alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a process reciting elements A, B and C specifically envisions embodiments consisting of, and consisting essentially of, A, B and C, excluding an element D that can be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter can define endpoints for a range of values that can be claimed for the parameter. For example, if Parameter X is exemplified herein to have value A and also exemplified to have value Z, it is envisioned that Parameter X can have a range of values from about A to about Z. Similarly, it is envisioned that disclosure of two or more ranges of values for a parameter (whether such ranges are nested, overlapping, or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X can have other ranges of values including 1-9,1-8,1-3,1-2,2-10,2-8,2-3,3-10,3-9, and so on.

When an element or layer is referred to as being “on,” “engaged to,” “connected to,” or “coupled to” another element or layer, it can be directly on, engaged, connected, or coupled to the other element or layer, or intervening elements or layers can be present. In contrast, when an element is referred to as being “directly on,” “directly engaged to,” “directly connected to” or “directly coupled to” another element or layer, there can be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. can be used herein to describe various elements, components, regions, layers and/or sections, these elements, components, regions, layers and/or sections should not be limited by these terms. These terms can be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, can be used herein for ease of description to describe one element or feature's relationship to another element(s) or feature(s) as illustrated in the figures. Spatially relative terms can be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device can be otherwise oriented (rotated 90 degrees or at other orientations) and the spatially relative descriptors used herein interpreted accordingly.

As used herein, the term “percutaneous” refers to something that is made, done, or effected through the skin.

As used herein, the term “percutaneous medical procedure” refers to accessing the internal organs or tissues via needle-puncture of the skin, rather than by using an open approach where the internal organs or tissues are exposed (e.g., typically with a scalpel).

As used herein, the term “non-vascular” when used with “percutaneous medical procedure” refers to a medical procedure performed on any portion of the subject's body distinct from the vasculature that is accessed percutaneously. Examples of percutaneous medical procedures can include a biopsy, a tissue ablation, a cryotherapy procedure, a brachytherapy procedure, an endovascular procedure, a drainage procedure an orthopedic procedure, a pain management procedure, a vertebroplasty procedure, a pedicle/screw placement procedure, a guidewire-placement procedure, a SI-Joint fixation procedure, a training procedure, or the like.

As used herein, the term “endovascular” when used with “percutaneous medical procedure” refers to a medical procedure performed on a blood vessel (or the lymphatic system) accessed percutaneously. Examples of endovascular percutaneous medical procedures can include an aneurism repair, a stent grafting/placement, a placement of an endovascular prosthesis, a placement of a wire, a catheterization, a filter placement, an angioplasty, or the like.

As used herein, the terms “interventional device” or “tracked instrument” refers to a medical instrument used during the non-vascular percutaneous medical procedure.

As used herein, the term “tracking system” refers to something used to observe one or more objects undergoing motion and supply a timely ordered sequence of tracking data (e.g., location data, orientation data, or the like) in a tracking coordinate system for further processing. As an example, the tracking system can be an electromagnetic tracking system that can observe an interventional device equipped with a sensor-coil as the interventional device moves through a patient's body.

As used herein, the term “tracking data” refers to information recorded by the tracking system related to an observation of one or more objects undergoing motion.

As used herein, the term “tracking coordinate system” refers to a three-dimensional (3D) Cartesian coordinate system that uses one or more numbers to determine the position of points or other geometric elements unique to the particular tracking system. For example, the tracking coordinate system can be rotated, scaled, or the like, from a standard 3D Cartesian coordinate system.

As used herein, the term “head-mounted device” or “headset” or “HIVID” refers to a display device, configured to be worn on the head, that has one or more display optics (including lenses) in front of one or more eyes. These terms can be referred to even more generally by the term “augmented reality system,” although it should be appreciated that the term “augmented reality system” is not limited to display devices configured to be worn on the head. In some instances, the head-mounted device can also include a non-transitory memory and a processing unit. Example of suitable head-mounted devices include various versions of the Microsoft HoloLens® mixed reality smart glasses.

As used herein, the terms “imaging system,” “image acquisition apparatus,” “image acquisition system” or the like refer to technology that creates a visual representation of the interior of a patient's body. For example, the imaging system can be a computed tomography (CT) system, a fluoroscopy system, a magnetic resonance imaging (MM) system, an ultrasound (US) system, or the like.

As used herein, the terms “coordinate system” or “augmented realty system coordinate system” refer to a 3D Cartesian coordinate system that uses one or more numbers to determine the position of points or other geometric elements unique to the particular augmented reality system or image acquisition system to which it pertains. For example, the headset coordinate system can be rotated, scaled, or the like, from a standard 3D Cartesian coordinate system.

As used herein, the terms “image data” or “image dataset” or “imaging data” refers to information recorded in 3D by the imaging system related to an observation of the interior of the patient's body. For example, the “image data” or “image dataset” can include processed two-dimensional or three-dimensional images or models such as tomographic images; e.g., represented by data formatted according to the Digital Imaging and Communications in Medicine (DICOM) standard or other relevant imaging standards.

As used herein, the terms “imaging coordinate system” or “image acquisition system coordinate system” refers to a 3D Cartesian coordinate system that uses one or more numbers to determine the position of points or other geometric elements unique to the particular imaging system. For example, the imaging coordinate system can be rotated, scaled, or the like, from a standard 3D Cartesian coordinate system.

As used herein, the terms “hologram”, “holographic,” “holographic projection”, or “holographic representation” refer to a computer-generated image projected to a lens of a headset. Generally, a hologram can be generated synthetically (in an augmented reality (AR)) and is not related to physical reality.

As used herein, the term “physical” refers to something real. Something that is physical is not holographic (or not computer-generated).

As used herein, the term “two-dimensional” or “2D” refers to something represented in two physical dimensions.

As used herein, the term “three-dimensional” or “3D” refers to something represented in three physical dimensions. An element that is “4D” (e.g., 3D plus a time and/or motion dimension) would be encompassed by the definition of three-dimensional or 3D.

As used herein, the term “integrated” can refer to two things being linked or coordinated. For example, a coil-sensor can be integrated with an interventional device.

As used herein, the term “degrees-of-freedom” or “DOF” refers to a number of independently variable factors. For example, a tracking system can have six degrees-of-freedom (or 6DOF), a 3D point and 3 dimensions of rotation.

As used herein, the term “real-time” refers to the actual time during which a process or event occurs. In other words, a real-time event is done live (within milliseconds so that results are available immediately as feedback). For example, a real-time event can be represented within 100 milliseconds of the event occurring.

As used herein, the terms “subject” and “patient” can be used interchangeably and refer to any organism to which a medical procedure can be applied, including various vertebrate organisms such as a human.

As used herein, the term “registration” refers to steps of transforming tracking data and body image data to a common coordinate system and creating a holographic display of images and information relative to a body of a physical patient during a procedure, for example, as further described in U.S. Patent Application Publication No. 2018/0303563 to West et al., and also applicant's co-owned U.S. patent application Ser. No. 17/110,991 to Black et al. and U.S. patent application Ser. No. 17/117,841 to Martin III et al., the entire disclosures of which are incorporated herein by reference.

The present technology relates to ways for providing holographic augmented reality visualization and guidance in performing a medical procedure on an anatomical site of a patient by a user. Systems and uses thereof can include an augmented reality system, a tracked instrument, an image acquisition system, and a computer system. The tracked instrument can include a sensor. The image acquisition system can be configured to acquire a holographic image dataset from the patient. The computer system can include a processor and a memory, where the computer system can be in communication with the augmented reality system, the tracked instrument, and the image acquisition system. The image acquisition system can actively acquire the holographic image dataset from the patient. The computer system can track the tracked instrument using the sensor to provide a tracked instrument dataset, where the computer system can register the holographic image dataset and the tracked instrument dataset with the patient. The augmented reality system can render a hologram based on the holographic image dataset from the patient for viewing by the user and can generate a feedback based on the holographic image dataset from the patient and the tracked instrument dataset. Such systems and uses thereof can accordingly provide at least one of visualization, guidance, and navigation of the tracked instrument to the user during the medical procedure in response to the feedback when the user performs a portion of the medical procedure on the patient while viewing the patient and the hologram with the augmented reality system.

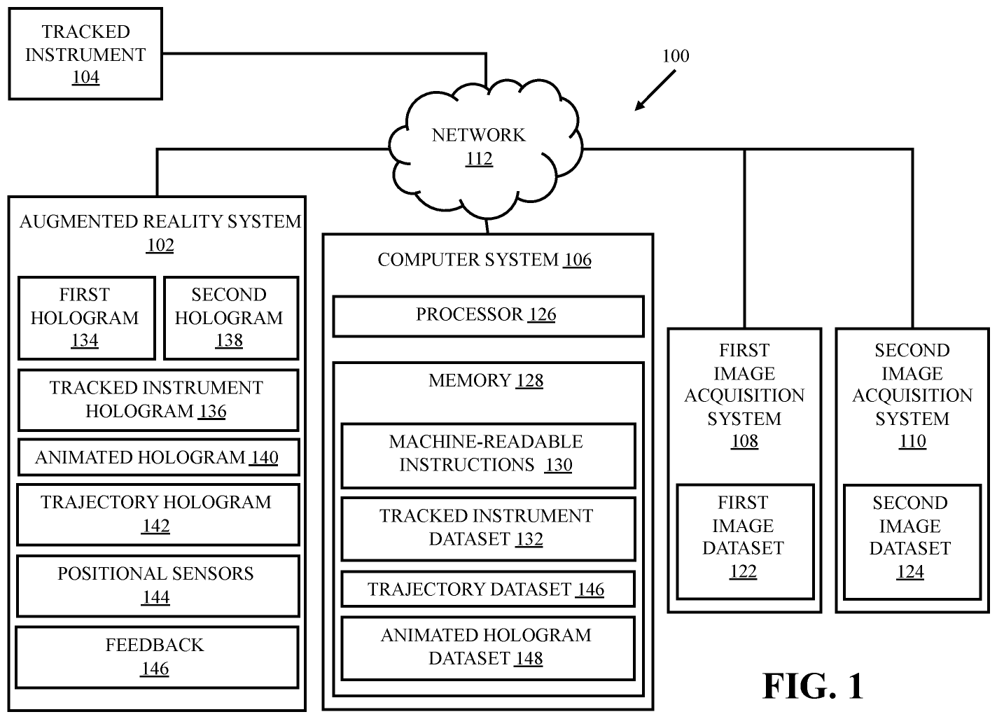

As shown in FIG. 1, a system 100 for holographic augmented reality visualization and guidance in performing a medical procedure on an anatomical site of a patient by a user includes an augmented reality system 102, a tracked instrument 104, a computer system 106, and a first image acquisition system 108. In certain embodiments, the system 100 can further include a second image acquisition system 110. Each of the augmented reality system 102, the tracked instrument 104, the first image acquisition system 108, and the second image acquisition system 110 can be selectively or permanently in communication with the computer system 106, for example, via a computer network 112. Other suitable instruments, tools, equipment, sub-systems, and the like for use with the holographic augmented reality visualization and guidance system 100, as well as other network means including wired and wireless means of communication between the components of the holographic augmented reality visualization and guidance system 100, can also be employed by the skilled artisan, as desired.

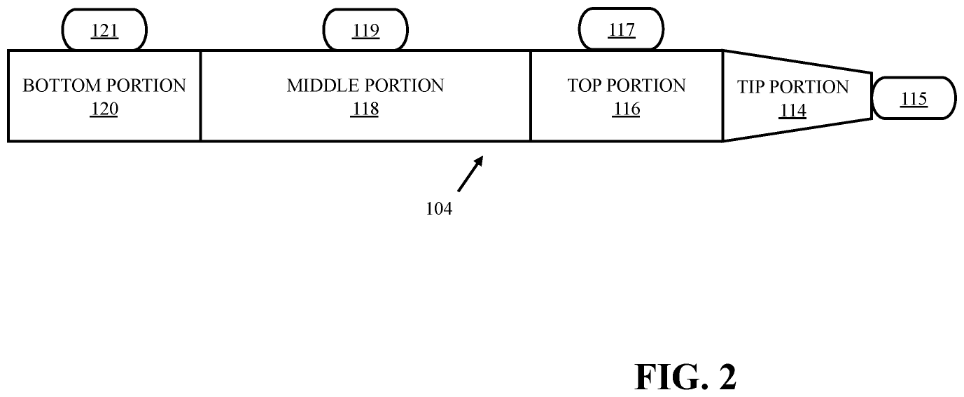

With reference to FIG. 2, the tracked instrument 104 is an interventional device that is sensorized so that that both a location and an orientation of the tracked instrument 104 can be determined by the computer system 106. In particular, the tracked instrument 104 can have an elongate body, such as long flexible tube, with a plurality of portions 114, 116, 118, 120 disposed along a length of the elongate body, which in turn can each have one of a plurality of sensors 115, 117, 119, 121. For example, the tracked instrument 104 can have a tip portion 114, a top portion 116, a middle portion 118, and a bottom portion 120. A tip sensor 115 can be disposed at the tip portion 114 of the tracked instrument 104. A top portion sensor 117 can be disposed at the top portion 116 of the tracked instrument 104. A middle portion sensor 119 can be disposed at the middle portion 118 of the tracked instrument 104. A bottom portion sensor 121 can be disposed at the bottom portion 120 of the tracked instrument 104. Each of the sensors 115, 117, 119, 121 can be in communication with or otherwise detectable by the computer system 106.

It should be appreciated that the tracking provided by the tip sensor 115 is especially advantageous as this can be used by the user as a preselected reference point for the tracked instrument 104. The preselected reference point can be configured to be an anchoring point for a trajectory hologram (shown in FIG. 1 and described herein as “142”) such as a holographic light ray that can be generated by the augmented reality system 102. The holographic light ray can assist the user with the alignment and movement of the tracked instrument 104 along a preferred pathway or trajectory, as described further herein. It should be appreciated that one skilled in the art can also select any number of preselected reference points, within the scope of this disclosure. In certain embodiments, the preselected reference point can be adjusted in real-time by the user during the medical procedure, and can alternatively be based on one or more of the other sensors 115, 117, 119, 121, as desired.

In certain examples, the sensors 115, 117, 119, 121 can be part of an electromagnetic (EM) tracking system that can be part of and/or used by the computer system 106 to detect the location and the orientation of a physical tracked instrument 104. For example, the sensors 115, 117, 119, 121 can include one or more sensor-coils. The computer system 106 can detect the one or more sensor-coils and provide tracking data (e.g., with six degrees of freedom) in response to the detection. For example, the tracking data can include real-time 3D position data and real-time 3D orientation data. The tracking system of the computer system 106 can also detect coil-sensors that are not located on the physical tracked instrument 104 or physical interventional device, such as one or more sensors located on fiducial markers or other imaging targets.

Further, the sensors 115, 117, 119, 121 can be configured to assess various additional information of the tracked instrument 104, such as angular velocity and acceleration of the tracked instrument 104. Nonlimiting examples of sensors 115, 117, 119, 121 suitable for determining angular velocity and acceleration include accelerometers, gyroscopes, electromagnetic sensors, and optical tracking sensors. Notably, use of electromagnetic sensors can enable more precise real-time object tracking of small objects without line-of-sight restrictions.

Other suitable tracking systems, such as optical tracking systems, can be used in conjunction with the augmented reality system 102 and the computer system 106. Embodiments where the tracked instrument 104 can communicate by transmission wirelessly or through a wired connection with the augmented reality system 102 and the computer system 106 are contemplated. It should also be appreciated that a skilled artisan can employ mixed types of sensors 115, 117, 119, 121, as desired.

Certain embodiments of the tracked instrument 104 can include the following aspects, which can depend on the type of medical procedure being performed, the anatomical site of the patient, and/or a particular step of the medical procedure being performed. Non-limiting examples include where the tracked instrument 104 includes a catheter, where the catheter can be configured to remove a fluid and/or deliver a fluid to an anatomical site, or where the catheter is a cardiac catheter, a balloon catheter, and/or a cardiac pacing or mapping catheter. Further non-limiting examples include where the tracked instrument 104 includes an orthopedic tool, including a saw, reamer, and other bone modification tools. Further non-limiting examples include where the tracked instrument 104 includes a tool used to install, adjust, or remove an implant, such as a mechanical heart valve, a biological heart valve, an orthopedic implant, a stent, and a mesh. Certain embodiments of the present technology can include where such implants themselves can be sensorized at least temporarily during the medical procedure to facilitate tracking of the same. Further non-limiting examples include where the tracked instrument 104 includes an ablation probe, such as a thermal ablation probe, including a radiofrequency ablation probe and a cyroablation probe. Further non-limiting examples include where the tracked instrument 104 includes a laparoscopic instrument, such as a laparoscope, inflator, forceps, scissors, probe, dissector, hook, and/or retractor. Further non-limiting examples include where the tracked instrument 104 includes other intervention tools, including powered and unpowered tools, various surgical tools, a needle, electrical probe, and a sensor, such as an oxygen sensor, pressure sensor, and an electrode. One of ordinary skill in the art can employ other suitable interventional devices for the tracked instrument 104, depending on the desired procedure or a particular step of the desired procedure, within the scope of the present disclosure.

With renewed reference to FIG. 1, the first image acquisition system 108 can be configured to acquire a first holographic image dataset 122 from the patient. In particular, the first image acquisition system 108 can be configured to acquire the first holographic image dataset 122 from the patient in a preoperative manner. In certain embodiments, the first image acquisition system 108 can include one or more of a magnetic resonance imaging (MM) apparatus, a computerized tomography (CT) apparatus, a projectional radiography apparatus, a positron emission tomography (PET) apparatus, and an ultrasound system. Other suitable types of instrumentation for the first image acquisition system 108 can also be employed, as desired. It is further possible to have the first image acquisition system 108 include multiple image acquisitions, including composite images, by the same or different imaging means, where the first image dataset 122 can therefore include multiple and/or composite images from the same or different imaging means.

Likewise, the second image acquisition system 110 can be configured to acquire a second holographic image dataset 124 from the patient. In particular, the second image acquisition system 110 can be configured to acquire the second holographic image dataset 124 from the patient in an intraoperative manner, and most particularly in real-time as the procedure is being undertaken. In certain embodiments, the second image acquisition system 110 can include one or more of an ultrasound system, including an ultrasound echocardiogram (ECG) imaging apparatus, a fluoroscopy apparatus, as well as other active or real-time imaging systems. Further embodiments include where the second holographic image dataset 124 can be acquired by a predetermined modality including one of a transthoracic echocardiogram (TTE), a transesophageal echocardiogram (TEE), and an intracardiac echocardiogram (ICE). Other suitable types of instrumentation and modalities for the second image acquisition system 110 can also be employed, as desired. It is further possible to have the second image acquisition system 110 include multiple image acquisitions, including composite images, by the same or different imaging means, where the second image dataset 124 can therefore include multiple and/or composite images from the same or different imaging means.

Although use of both the first image acquisition system 108 and the second image acquisition system 110 is shown and described herein, embodiments in which only one or the other of the first image acquisition system 108 and the second image acquisition system 110 is employed, are considered to be within the scope of the present disclosure.

With reference to FIG. 1, the computer system 106 of the present disclosure can include a processor 126 configured to perform functions associated with the operation of the system 100 for holographic augmented reality visualization and guidance. The processor 126 can include one or more types of general or specific purpose processors. In certain embodiments, multiple processors 126 can be utilized. The processor 126 can include one or more of general-purpose computers, special purpose computers, microprocessors, digital signal processors (DSPs), field-programmable gate arrays (FPGAs), application-specific integrated circuits (ASICs), and processors based on a multi-core processor architecture, as non-limiting examples.

With continued reference to FIG. 1, the computer system 106 of the present disclosure can include a memory 128 on which tangible, non-transitory, machine-readable instructions 130 can be stored. The memory 128 can include one or more types of memory and can include any type suitable to the local application environment. Examples include where the memory 128 can include various implementations of volatile and/or nonvolatile data storage technology, such as a semiconductor-based memory device, a magnetic memory device and system, an optical memory device and system, fixed memory, and removable memory. For example, the memory 128 can include one or more of random access memory (RAM), read only memory (ROM), static storage such as a magnetic or optical disk, hard disk drive (HDD), or any other type of non-transitory machine or computer readable media, as well as combinations of the aforementioned types of memory. Instructions stored in the memory 128 can include program instructions or computer program code that, when executed by the processor 126, enables the system 100 for holographic augmented reality visualization and guidance to perform tasks as described herein.

The machine-readable instructions 130 can include one or more various modules. Such modules can be implemented as one or more of functional logic, hardware logic, electronic circuitry, software modules, and the like. The modules can include one or more of an augmented reality system module, an image acquiring module, an instrument tracking module, an image dataset registering module, a hologram rendering module, an image registering module, a trajectory hologram rendering module, and/or other suitable modules, as desired.

The computer system 106 can be in communication with the augmented reality system 102, the tracked instrument 104, and the first image acquisition system 108, and the second image acquisition system 110, for example, via the network 112, and can be configured by the machine-readable instructions 130 to operate in accordance with various methods for holographic augmented reality visualization and guidance in performing a medical procedure on an anatomical site of a patient by a user as described further herein. The computer system 106 can be separately provided and spaced apart from the augmented reality system 102, or the computer system 106 can be provided together with the augmented reality system 102 as a singular one-piece unit or integrated with other systems, as desired.

It should be appreciated that the network 112 of the system 100 for holographic augmented reality visualization and guidance can include various wireless and wired communication networks, including a radio access network, such as LTE or 5G, a local area network (LAN), a wide area network (WAN) such as the Internet, or wireless LAN (WLAN), as non-limiting examples. It will be appreciated that such network examples are not intended to be limiting, and that the scope of this disclosure includes implementations in which one or more computing platforms of the holographic augmented reality visualization and guidance system 100 can be operatively linked via some other communication coupling, including combinations of wireless and wired communication networks. One or more components and subcomponents of the system 100 can be configured to communicate with the networked environment via wireless or wired connections. In certain embodiments, one or more computing platforms can be configured to communicate directly with each other via wireless or wired connections. Examples of various computing platforms and networked devices include, but are not limited to, smartphones, wearable devices, tablets, laptop computers, desktop computers, Internet of Things (IoT) devices, or other mobile or stationary devices such as standalone servers, networked servers, or an array of servers.

In certain embodiments, the computer system 106 can be configured to track the tracked instrument 104 using the plurality of sensors 115, 117, 119, 121 to provide a tracked instrument dataset 132. The tracked instrument dataset 132 can be stored using the memory 128. In particular, the tracked instrument dataset 132 can include the location and the orientation of the tracked instrument 104 in physical space, for example. The computer system 106 can also be configured to register the first holographic image dataset 122 from the first image acquisition system 108 and the tracked instrument dataset 132 obtained by the computer system 106 with the patient, as also described herein.

With continued reference to FIG. 1, the augmented reality system 102 can be configured to render a plurality of holograms 134, 136, 138, 140, 142 in operation of the system 100 in accordance with the present disclosure. In particular, the augmented reality system 102 can include a mixed reality (MR) display such as one or more MR smart glasses or MR head-mounted displays. Further nonlimiting examples of the augmented reality system 102 can include the Magic Leap One® or versions of the Microsoft HoloLens®. It should be appreciated that other types of MR displays can be used for the augmented reality system 102, as long as they are capable of superimposing computer-generated imagery, including holograms, over real-world objects. Additionally, although the augmented reality system 102 can be described primarily as including a head-mounted display, it should be understood that other types of displays that are not head-mounted, but which are capable of generating and superimposing holograms 134, 136, 138, 140 over real-world views, can also be employed, as desired.

In certain embodiments of the system 100, the augmented reality system 102 and the computer system 106 can be integrated into either a single component or multiple shared components. For example, the computer system 106 can be onboard or integrated into a mixed reality display such as smart glasses or a headset. The augmented reality system 102 and the computer system 106 can also be separate components that communicate through a local network 112 or where the computer system 106 is remote from the augmented reality system 102, including where the computer system 106 is cloud based, for example. It should be appreciated that in instances where the augmented reality system 102 is not integrated with or does not contain the computer system 106, the augmented reality system 102 can further include an additional non-transitory memory and a processing unit (that can include one or more hardware processors) that can aid in the rendering or generation of holograms 134, 136, 138, 140, 142. The augmented reality system 102 can also include a recording means or camera to record one or more images, one or more image-generation components to generate/display a visualization of the holograms 134, 136, 138, 140, 142, and/or other visualization and/or recording elements. Likewise, the augmented reality system 102 can transmit images, recordings, and/or videos of one or more nonaugmented views, holograms 134, 136, 138, 140, 142, and/or mixed reality views to the computer system 106 for storage or recording, whether the computer system 106 is local or remote from the augmented reality system 102.

It should be appreciated that in certain embodiments the augmented reality system 102 can also include one or more positional sensors 144. One or more positional sensors 144 of the augmented reality system 102 can be configured to determine various positional information for the augmented reality system 102, such as the approximated position in three-dimensional (3D) space, the orientation, angular velocity, and acceleration of the augmented reality system 102. For example, it should be understood that this can allow the holographic imagery to be accurately displayed within the field of view of the user, in operation. Nonlimiting examples of the of positional sensors 144 include accelerometers, gyroscopes, electromagnetic sensors, and/or optical tracking sensors. It should further be appreciated that a skilled artisan can employ different types and numbers of positional sensors 144 of the augmented reality system 102, for example, as required by the procedure or situation within which the augmented reality system 102 is being used.

As shown in FIG. 1, for example, the holograms 134, 136, 138, 140, 142 generated by the augmented reality system 102 can include one or more of a first hologram 134, a tracked instrument hologram 136, a second hologram 138, an animated hologram 140, and a trajectory hologram 142. The first hologram 134 generated by the augmented reality system 102 can be based on the first holographic image dataset 122 from the patient. The tracked instrument hologram 136 generated by the augmented reality system 102 can be based on the tracked instrument dataset 132. The second hologram 138 generated by the augmented reality system 102 can be based on the second holographic image dataset 124. The animated hologram 140 can be based on a processing by the computer system 106 of the second holographic image dataset 124 to provide an animated hologram dataset 148, as described herein. The trajectory hologram 142 can be based on a trajectory dataset 146, which can be either manually or automatically selected and stored in the memory 128 of the computer system 106, as described herein.

The augmented reality system 102 can also be configured to, in addition to rendering or generating the various holograms 134, 136, 138, 140, 142, show various operating information or details to the user. For example, the augmented reality system 102 can project the operating information within a field of view of the user, adjacent to various real-world objects, as well as overlaid upon or highlighting real-world objects, such as one or more portions of the anatomical site of the patient, the tracked instrument 104, or the various holograms 134, 136, 138, 140, 142. The operating information can include real-time navigation instructions or guidance for the trajectory to be employed, for example. It should be appreciated that the augmented reality system 102 can project the operating information over various real-world objects such as the tracked instrument 104, as well as over the various holograms 134, 136, 138, 140, 142 rendered, as desired. Generation of such operating information or details allows the user to simultaneously view the patient and the plurality of operating information in the same field of view. Also, generation of the operating information or details together with the various holograms 134, 136, 138, 140, 142 permits the user to plan, size, or pre-orient the tracked instrument 104, in operation.

As shown in FIG. 1, the computer system 106 can be in communication with the augmented reality system 102 and the tracked instrument 104. The computer system 106 can be configured to store and generate the operating information, either through manual intervention by the user and/or other medical professionals or automatically based on machine-readable instructions 130 encoded within the memory 128. For example, the operating information can be generated in the augmented reality system 102 depending on a sensor-determined position and/or orientation of the tracked instrument 104, such as by using algorithms, artificial intelligence (AI) protocols, or other user-inputted data or thresholds. In addition, the computer system 106 can be further configured to permit the user to selectively adjust the operating information in real-time. For example, the user can adjust the position or orientation of the trajectory hologram 142. In addition, the user can decide which of the operating information or data is actively being shown. It should be appreciated that other settings and attributes of the operating information can be adjusted by the user in real-time, within the scope of this disclosure.

With respect to using the system 100 for holographic augmented reality visualization and guidance in performing a medical procedure, it should be understood that the augmented reality system 102 advantageously permits the user to perform the medical procedure while viewing the patient and the first hologram 134, and optionally the instrument hologram 136, with the augmented reality system 102, as well as selectively viewing any of the holograms 134, 136, 138, 140, 142 generated thereby. Likewise, the user is advantageously permitted to employ the augmented reality system 102 for at least one of visualization, guidance, and navigation of the tracked instrument 104 during the medical procedure, as described herein with respect to various ways of using the system 100.

In certain embodiments, the trajectory hologram 142 can include a holographic light ray illustrating the predetermined trajectory of the tracked instrument 104, for example. The holographic light ray can be linear or curvilinear, can have one or more angles, and/or can depict an optimum path for the tracked instrument 104. The trajectory hologram 142 can also be used to clearly identify various aspects related to a particular medical procedure and/or particular anatomical site of the patient. For example, the trajectory hologram 142 can display a percutaneous entry point on the patient and an intravascular landing point within the patient for the tracked instrument 104, such as a preferred landing zone with the structure of the heart of the patient for an implant to be deployed, in certain cardiac medical procedures. It should be appreciated that the overall size, shape, and/or orientation of the trajectory hologram 142 generated by the augmented reality system 102 can be based on operating information from the computer system 106 including preoperative data and intraoperative data, which can be particular to a given medical procedure and/or particular to a given tracked instrument 104. Various types of preoperative data and intraoperative data can be adaptable to a variety of medical procedures, however. It should also be appreciated that the operating information can include additional data from other sensors in the operating arena and also the other holographic projections 134, 136, 138, 140 being generated by the augmented reality system 102.

Preoperative data can include information related to the patient obtained prior to the medical procedure, for example, using the first holographic image acquisition system 108 as well as data obtained, processed, and/or annotated from a variety of sources. Embodiments of preoperative data include various images, composite images, annotated images, as well as one or more markers or flagged points or portions of the anatomical site of the patient. Certain nonlimiting examples of preoperative data include static images or recordings from a transesophageal echocardiogram, a transabdominal echocardiograph, a transthoracic echocardiogram, a computerized tomography (CT) scan, a magnetic resonance imaging (MM) scan, or an X-ray. It should be appreciated that the preoperative data can include information from other diagnostic medical procedures, imaging modalities, and modeling systems, as desired.

Intraoperative data can include information related to the patient and the anatomical site of the patient obtained in real-time, including during the medical procedure, for example, using the second holographic image acquisition system 110. For example, the diagnostic medical procedures listed herein with respect to the preoperative data can be performed simultaneously with the current medical procedure and collected and used in real time as intraoperative data. For example, a real time ultrasound image can be obtained and integrated into the second holographic image acquisition system 110, which can provide a real time view, static or movable in real time, in conjunction with the second holographic image acquisition system 110.

Operating information as used in the present technology can further include composite or fused preoperative and intraoperative data. Composite preoperative and intraoperative data can include a merger of preoperative data and intraoperative data in such a way to present more concise and approximated images and animations to the user. In certain instances, the fusion of data can be performed in manual fashion. In other instances, the fusion of data can be done by the computer system 106, for example, using one or more algorithms set forth in the machine-readable instructions 130 or via artificial intelligence (AI).

With reference again to the augmented reality system 102 and the trajectory hologram 142, use of the holographic light ray can include various aspects. In certain embodiments, the holographic light ray can be anchored on the preselected reference point of the tracked instrument 104. The intended trajectory can also be adjusted via the computer system 106 in real-time by the user, for example, to address an unforeseen complication that arises during the medical procedure. It is believed that the trajectory hologram 142, along with other holographic projections, can minimize a risk of complications associated with certain medical procedures; e.g., transapical approach procedures. For example, an overall size of an incision in the heart, arteries, or veins can be minimized because the user is able to be more precise with the intended trajectory of the tracked instrument 104 via the trajectory hologram 142, such as the holographic light ray. As another example, it is believed that the trajectory hologram 142 can permit the user to more easily find an optimal approach angle in using a given tracked instrument 104 in a particular medical procedure, such as for a valve implantation or a paravalvular leak (PVL) closure. Also, by enabling the user to more easily find the optimal approach angle, the user can better avoid critical structures; e.g., lung tissue, coronary arteries, and the left anterior descending artery during cardiac procedure.

Aspects of the present technology can be further appreciated in situations where a holographic display of a real-time intraoperative scan can be overlaid with a holographic display of a preoperative scan. Composite or fused preoperative and intraoperative data, for example, can include a holographic fusion of CT scan images and intraoperative fluoroscopic imaging, thereby modeling the anatomical site of the patient; e.g., heart motion associated with cardiac cycle. What is more, composite preoperative and intraoperative data can further include overlays that notify or warn the user of sensitive areas in the body of the patient that should not come into contact with the tracked instrument 104. It should be appreciated that different applications of the composite preoperative and intraoperative data can be employed by one skilled in the art, within the scope of this disclosure.

In certain embodiments, the computer system 106, as part of the system 100 for holographic augmented reality visualization and guidance, can be configured to predict a shape of an implant involved in the medical procedure. For example, the shape, including the location and position (e.g., orientation), of a valve can be predicted once the implant has been deployed by the tracked instrument 104. The predicted shape of the implant can also be visualized in the form of a hologram further generated by the augmented reality system 102, for example. In certain embodiments, the computer system 106 can be configured to facilitate a co-axial deployment, e.g., a centering of a valve within the endovascular structure, with the tracked instrument 104. The augmented reality system 102 can be employed to generate a notification in the form of “error bars” or provide coloration (e.g., “green” for acceptable, and “red” for unacceptable) to guide the user in the co-axial deployment during the medical procedure.

In certain embodiments, the computer system 106 can be employed to predict a remodeling of the anatomical site of the patient (e.g., endovascular or heart structure) that is expected to result from the medical procedure (e.g., relative to a deployed position of an implant) over time. In particular, the computer system 106 can project or predict how the anatomical site (e.g., heart muscle, bone, soft tissue, etc.) will be remodeled over time with a particular implant placement, and thus permit for planning of the implant placement in a manner that will minimize the remodeling that can occur over time. The computer system 106 can also be used to assist with size selection of a prosthesis or implant prior to completion of the medical procedure. The employment of the system 100 for holographic augmented reality visualization and guidance to select appropriate sizing can minimize an opportunity for patient-prosthesis mismatch (PPM), which can otherwise occur when an implanted prosthetic (e.g., heart valve) is either too small or large for the patient.

It should also be appreciated the system 100 can permit the user to customize how much operating information is displayed by the augmented reality system 102. The user can customize the settings and attributes of the operating information using, for example, the computer system 106. The system 100 allows the user to perform an instrument insertion during the medical procedure at any desired angle and without the need for additional physical instrument guides.

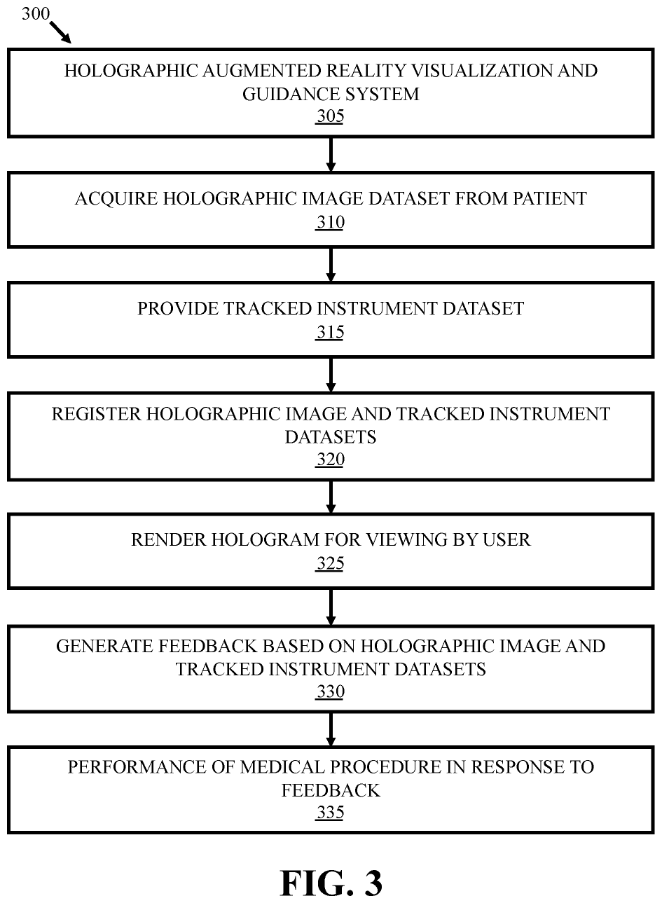

FIG. 3 illustrates an example flow diagram of a method 300 for holographic augmented reality visualization and guidance in performing a medical procedure on an anatomical site of a patient by a user, according to an embodiment of the present technology. It should be understood that the general outline of the method 300 can employ the various systems as described herein. Furthermore, the method 300 can include the use of additional components and subcomponents thereof, as well as additional steps and subprocesses, as described herein.

With respect to the holographic augmented reality visualization and guidance system provided in step 305, the system can include the augmented reality system, the tracked instrument having a sensor, the image acquisition system, and the computer system. The image acquisition system can be configured to acquire the holographic image dataset from the patient. The computer system can include the processor and the memory, where the computer system is in communication with the augmented reality system, the tracked instrument, and the image acquisition system. With respect to step 310, the image acquisition system can be used to acquire the holographic image dataset from the patient. With respect to step 315, the computer system can be used to track the tracked instrument using the sensor to provide a tracked instrument dataset. With respect to step 320, the computer system can be used to register the holographic image dataset and the tracked instrument dataset with the patient. With respect to step 325, the augmented reality system can be used to render a hologram based on the holographic image dataset from the patient for viewing by the user. With respect to step 330, the augmented reality system can be used to generate a feedback based on the holographic image dataset from the patient and the tracked instrument dataset. With respect to step 335, the user can perform a portion of the medical procedure on the patient while viewing the patient and the hologram with the augmented reality system. In this way, the user can employ the augmented reality system for at least one of visualization, guidance, and navigation of the tracked instrument during the medical procedure in response to the feedback.

With respect to generating the feedback based on the holographic image dataset from the patient and the tracked instrument dataset using the augmented reality system, the feedback can include the following aspects. Various types and combinations of feedback can be used. For example, the feedback can include one or more of a visual notification, an auditory notification, and a data notification to the user. Where a visual notification is provided, various types of visual cues, colors, images, text, and symbols can be employed. Embodiments include where the visual notification can be provided as part of the hologram rendered by the augmented reality system.

Feedback can be generated following a projected performance, by the user, of the portion of the medical procedure on the patient using the tracked instrument. For example, the user can place the tracked instrument in various positions, including various locations and/or orientations, where the projected performance of the tracked instrument can be displayed at one or more of such positions. In this way, the user can ascertain the projected performance of using the tracked instrument in various ways without actually performing the portion of the medical procedure. The projected performance can also be determined preoperatively with respect to the medical procedure. It is therefore possible to provide feedback to the user of various insertion routes of the tracked instrument into the anatomical site of the patient prior to initiating the medical procedure and inserting the tracked instrument into the patient.

In certain embodiments, the projected performance can be determined by planning using the computer system and rendering using the augmented reality system. A predetermined trajectory of insertion of the tracked instrument into the anatomical site of the patient can be planned by the computer system in order to provide a predetermined trajectory dataset. The augmented reality system can then render a trajectory hologram based on the predetermined trajectory dataset. In this way, the user can see an effect or result of performing the portion of the medical procedure without actually doing so, where conflicts, identification of interfering structure, and/or undesired effects on the anatomical site of the patient can be minimized prior to taking action in the real world. Certain embodiments include where the trajectory hologram can be configured as a holographic light ray illustrating the predetermined trajectory of the tracked instrument. Various types of projected performance can be rendered by the augmented reality system, where nonlimiting examples include having the projected performance indicative of a projected treatment zone by the tracked instrument, having the projected performance indicative of a projected implant placement by the tracked instrument, and having the projected performance indicative of a projected insertion of the tracked instrument into the anatomical site of the patient. For example, where a projected treatment zone is displayed, the user can attenuate the size of the projected treatment zone based upon a setting of the tracked instrument. Multiple sizes of various treatment zones can therefore be displayed at the same time (e.g., concentric ablation zones) and the user can select a setting of the tracked instrument based upon a desired size or shape of a treatment zone.

In certain embodiments, the present technology can generate the feedback during the performance of the portion of the medical procedure on the patient by the user. For example, the feedback can be generated in real time while the user is performing one or more portions of the medical procedure at the anatomical site of the patient. The feedback can include a notification to the user to proceed with performance of the portion of the medical procedure, to pause performance of the portion of the medical procedure, and/or to cease performance of the portion of the medical procedure. Where the notification is a visual notification comprised by the hologram rendered by the augmented reality system, for example, the visual notification can include one or more color changes, shape changes, images, text, and symbols with respect to the hologram.

Ways of using the present systems for holographic augmented reality visualization and guidance in performing a medical procedure on an anatomical site of a patient by a user can employ another or second image acquisition system. The second image acquisition system can be configured to acquire a second holographic image dataset from the patient and the computer system can be in communication with the second image acquisition system. Methods can therefore include acquiring, by the second image acquisition system, the second holographic image dataset from the patient. Such methods can further include registering, by the computer system, the second holographic image dataset with the patient and rendering, by the augmented reality system, a second hologram based on the second holographic image dataset from the patient. In this way, for example, the holographic image dataset from the patient can be preoperative and the second holographic image dataset can be intraoperative and acquired in real-time during the medical procedure.

Methods of the present technology can also include the following aspects. It is possible to generate, by the computer system and based on the second holographic image dataset acquired in real-time, an animated hologram dataset relative to a predetermined portion of one of the hologram, the second hologram, and the hologram and the second hologram. The augmented reality system can then be used to render an animated hologram from the animated hologram dataset for viewing by the user during the medical procedure. The computer system can be used to select the predetermined portion of one of the hologram, the second hologram, and the hologram and the second hologram to be animated. Examples include where the image acquisition system includes a magnetic resonance imaging (MM) apparatus and/or a computerized tomography (CT) apparatus and the second image acquisition system includes an ultrasound apparatus.

In certain embodiments, ways for holographic augmented reality visualization and guidance in performing a medical procedure can include using the computer system to record the holographic image dataset, the tracked instrument dataset, the hologram, the feedback, and/or a view of the patient and the hologram. In this way, the computer system can be configured to record user performance of the medical procedure following the generation of the feedback. Likewise, the computer system can be configured to record aspects of the performance of the medical procedure on the anatomical site of the patient by the user.

Where the present technology records aspects of the medical procedure, the recording can be used track certain actions and outcomes of portions of the medical procedure that can be used in real time analysis as well as post-procedure analysis and evaluation. Recording can include tracking one or more steps or actions of a medical procedure, movement of one or more surgical instruments, and the anatomy of a patient (pre- and post-intervention) in real-time within a three-dimensional space. The recording and tracking can be used to generate real-time feedback to the user, which can be based on a comparison of a real-world position relative to a holographic guidance trajectory or treatment zone. Post-operative assessment of the medical procedure based upon the recording of the tracked instrument, anatomical site of the patient, and performance by the user is possible. Currently, effectuation and assessment of surgical procedures and outcomes may be tied to peer-reviewed scientific literature, but no quantitative bridge exists between the specifics of procedures, such as location, accuracy, and therapy, to peer reviewed outcomes or complications. Outcome predictions are, in certain instances, based entirely on a few points from taken in a given surgical procedure, which can be determined using methods such as post-operative imaging and an operative report. The present technology can afford assessment of three-dimensional hologram renderings and tracked instruments to provide new ways of quantifying certain actions with outcomes.

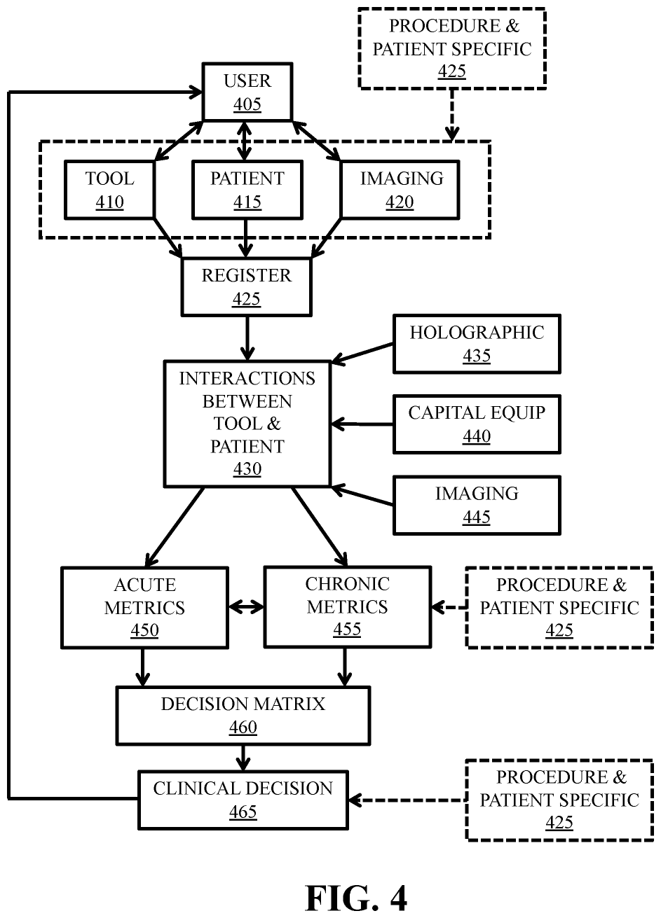

FIG. 4 is a schematic illustration of system components and process interactions showing ways to provide holographic augmented reality visualization and guidance in performing a medical procedure. The user 405, including a medical practitioner such as a surgeon, can select one or more tools 410, including one or more types of various tracked instruments 104, appropriate for the particular medical procedure to be conducted on a particular anatomical site of the patient 415. Likewise, the imaging 420 employed can be dependent on the one or more tools 410 and the anatomy of the patient 415, where the imaging 420 can include use of one or more image acquisition systems 108, 110. It can therefore be seen the tool(s) 410, anatomical site of the patient 415, and imaging 420 can be specific to the intended medical procedure and the patient, as indicated at 425.

Imaging 420 can include use of an image acquisition system 108, 110 configured to acquire a holographic image dataset 122, 124 from the patient 415. With reference back to FIG. 1, a computer system 106 can be configured to track the tool(s) 410 (e.g., tracked instrument(s) 104) using a sensor associated therewith to provide a tracked instrument dataset, where the computer system 106 can register the holographic image dataset and the tracked instrument dataset with the patient, as shown at 425. In this way, interactions between the tool(s) 410 (e.g., tracked instrument(s) 104) and the patient 415 can be determined at 430, where the augmented reality system 102 (see FIG. 1) can render a hologram based on the holographic image dataset from the patient 415 for viewing by the user 405. One or more rendered holograms can be provided as holographic information 435 in conjunction with various imaging systems 445 and/or data provided by capital equipment 440 used in the medical procedure.

Various metrics, including acute metrics 450 and chronic metrics 455, relative to the medical procedure can be determined relative to the interaction between the tool and the patient 430 as employed by the user 405. Such metrics can likewise be procedure and patient specific 425. For example, tumor ablation can be particular to location, size, and nearby structure of the anatomical site in a particular patient. Other metrics can be related to common landmarks or fiducials, for example, for installation of an implant at an anatomical site in the patient, but where local topology and patient specific morphology based on various imaging means can be used to adapt an established procedure for the particular patient 415. These metrics can be provided as feedback to guide the user 405 in performance of the medical procedure and/or can be recorded and tracked for post-operative analysis.

The acute metrics 450 and/or chronic metrics 455, in conjunction with holographic 435, capital equipment 440, and/or imaging 445 data, and/or the interactions between the tool(s) and the patient 430 can be used independently or in combination in generation of feedback to the user 405. Such feedback can include one or more notifications to the user 405 to proceed with performance of the portion of the medical procedure, to pause performance of the portion of the medical procedure, or to cease performance of the portion of the medical procedure, for example. Such notifications can be part of a predetermined decision matrix 460 that informs the user 405 of options and/or projected outcomes in performing a portion of the medical procedure.

The user 405 can therefore make a clinical decision 465 relative to the medical procedure based upon the feedback presented by the interactions between the tool and the patient 430, including any holographic 435, capital equipment 440, and imaging 445 data, as well as consideration of acute metrics 450 and chronic metrics 455. Upon making the clinical decision 465, the user 405 can perform an action using the tool 410 on the anatomical site of the patient, as informed by the feedback. It should be recognized that the clinical decision 465 can be procedure and patient specific, as indicated at 425. The user 405 can then continue to a subsequent step of the medical procedure taking one or more of the same considerations and feedback into account. As such, the present technology can therefore provide feedback at multiple stages of medical procedure, the process continuing recursively or in a loop until the medical procedure is determined to have reached completion.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments can be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A method for holographic augmented reality visualization and guidance in performing a medical procedure on an anatomical site of a patient by a user, comprising:
providing a system including:
an augmented reality system,
a tracked instrument having a sensor,
an image acquisition system configured to acquire a holographic image dataset from the patient, and
a computer system having a processor and a memory, the computer system in communication with the augmented reality system, the tracked instrument, and the image acquisition system;
acquiring, by the image acquisition system, the holographic image dataset from the patient;
tracking, by the computer system, the tracked instrument using the sensor to provide a tracked instrument dataset;
registering, by the computer system, the holographic image dataset and the tracked instrument dataset with the patient;
rendering, by the augmented reality system, a hologram based on the holographic image dataset from the patient for viewing by the user;
generating, by the augmented reality system, a feedback based on the holographic image dataset from the patient and the tracked instrument dataset; and
performing, by the user, a portion of the medical procedure on the patient while viewing the patient and the hologram with the augmented reality system, whereby the user employs the augmented reality system for at least one of visualization, guidance, and navigation of the tracked instrument during the medical procedure in response to the feedback.

2. The method of claim 1, wherein the feedback includes a member selected from a group consisting of: a visual notification; an auditory notification; a data notification; and
combinations thereof.

3. The method of claim 2, wherein the visual notification is comprised by the hologram rendered by the augmented reality system.

4. The method of claim 1, wherein the feedback is generated following a projected performance, by the user, of the portion of the medical procedure on the patient using the tracked instrument.

5. The method of claim 4, wherein the projected performance is determined preoperatively with respect to the medical procedure.

6. The method of claim 4, wherein the projected performance is determined by steps of:
planning, by the computer system, a predetermined trajectory of insertion of the tracked instrument into the anatomical site of the patient to provide a predetermined trajectory dataset; and
rendering, by the augmented reality system, a trajectory hologram based on the predetermined trajectory dataset.

7. The method of claim 6, wherein the trajectory hologram is a holographic light ray illustrating the predetermined trajectory of the tracked instrument.

8. The method of claim 4, wherein the projected performance is indicative of a projected treatment zone by the tracked instrument.

9. The method of claim 4, wherein the projected performance is indicative of a projected implant placement by the tracked instrument.

10. The method of claim 4, wherein the projected performance is indicative of a projected insertion of the tracked instrument into the anatomical site of the patient.

11. The method of claim 1, wherein the feedback is generated during the performing, by the user, of the portion of the medical procedure on the patient.

12. The method of claim 11, wherein the feedback includes a notification to the user to proceed with performance of the portion of the medical procedure, to pause performance of the portion of the medical procedure, or to cease performance of the portion of the medical procedure.

13. The method of claim 12, wherein the notification is a visual notification comprised by the hologram rendered by the augmented reality system.

14. The method of claim 1, wherein the system further includes another image acquisition system configured to acquire another holographic image dataset from the patient and the computer system is in communication with the another image acquisition system.

15. The method of claim 14, further comprising:
acquiring, by the another image acquisition system, the another holographic image dataset from the patient;
registering, by the computer system, the another holographic image dataset with the patient; and
rendering, by the augmented reality system, another hologram based on the another holographic image dataset from the patient,
wherein the holographic image dataset from the patient is preoperative and the another holographic image dataset is intraoperative and acquired in real-time during the medical procedure.

16. The method of claim 15, further comprising:
generating, by the computer system and based on the another holographic image dataset acquired in real-time, an animated hologram dataset relative to a predetermined portion of one of the hologram, the another hologram, and the hologram and the another hologram; and
rendering, by the augmented reality system, an animated hologram from the animated hologram dataset for viewing by the user during the medical procedure.

17. The method of claim 16, further comprising selecting, by the computer system, the predetermined portion of one of the hologram, the another hologram, and the hologram and the another hologram to be animated.

18. The method of claim 15, wherein the image acquisition system includes one of a magnetic resonance imaging (MM) apparatus and a computerized tomography (CT) apparatus and the another image acquisition system includes an ultrasound apparatus.

19. The method of claim 1, further comprising recording, using the computer system, a member selected from a group consisting of: the holographic image dataset; the tracked instrument dataset; the hologram; the feedback; a view of the patient and the hologram; and combinations thereof.

20. A system for holographic augmented reality visualization and guidance in performing a medical procedure on an anatomical site of a patient by a user, comprising:
an augmented reality system;
a tracked instrument having a sensor;
an image acquisition system configured to acquire a holographic image dataset from the patient; and
a computer system having a processor and a memory, the computer system in communication with the augmented reality system, the tracked instrument, and the image acquisition system,
wherein:
the image acquisition system is configured to acquire the holographic image dataset from the patient;
the computer system is configured to track the tracked instrument using the sensor to provide a tracked instrument dataset, and is configured to register the holographic image dataset and the tracked instrument dataset with the patient;
the augmented reality system is configured to render a hologram based on the holographic image dataset from the patient for viewing by the user, and is configured to generate a feedback based on the holographic image dataset from the patient and the tracked instrument dataset; and
the system thereby configured to provide at least one of visualization, guidance, and navigation of the tracked instrument to the user during the medical procedure in response to the feedback when the user performs a portion of the medical procedure on the patient while viewing the patient and the hologram with the augmented reality system.
